(19) United States
(12) Patent Application Publication
(10) Pub. No.: US 2024/0026424 A1
(43) Pub. Date: Jan. 25, 2024

(54) REAGENT AND METHOD FOR DETECTING TARGET NUCLEIC ACID USING SAME

(71) Applicant: CANON KABUSHIKI KAISHA, Tokyo (JP)

(72) Inventors: TAKUSHI ICHINOO, Tokyo (JP); SATOSHI OGAWA, Kanagawa (JP); MASATO MINAMI, Kanagawa (JP); Tetsuya Yano, Ibaraki (JP)

(21) Appl. No.: 18/478,721

(22) Filed: Sep. 29, 2023

Related U.S. Application Data
(63) Continuation of application No. PCT/JP2022/015771, filed on Mar. 30, 2022.

(30) Foreign Application Priority Data
Apr. 2, 2021 (JP) 2021-063492
Feb. 2, 2022 (JP) 2022-015061

(51) Int. Cl. C12Q 1/6818; C12Q 1/6806; C12N 9/22; C12N 15/11; G01N 21/64

(52) U.S. Cl. CPC C12Q 1/6818; C12Q 1/6806; C12N 9/22; C12N 15/11; G01N 21/6428; C12N 2310/20; C12N 2320/10; G01N 2021/6439

(57) ABSTRACT

A reagent contains complex particles, each including a particle and a complex bound to the particle, and an aqueous liquid in which the complex particles are dispersed. The complex includes a Cas protein and a guide RNA bound to the Cas protein.

### CROSS-REFERENCE TO RELATED APPLICATIONS

This application is a Continuation of International Patent Application No. PCT/JP2022/015771, filed Mar. 30, 2022, which claims the benefit of Japanese Patent Application No. 2021-063492, filed Apr. 2, 2021 and No. 2022-015061, filed Feb. 2, 2022, all of which are hereby incorporated by reference herein in their entirety.

### TECHNICAL FIELD

The present invention relates to a reagent and a method for detecting a target nucleic acid using the reagent.

### BACKGROUND ART

Jennifer Doudna and her colleagues at the University of California have reported highly sensitive detection of target DNA using a trans-cleavage reaction of a genome-editing enzyme, Cas12a. A complex made up of Cas12a, a type of Cas protein (CRISPR-associated protein), and crRNA (CRISPR RNA, which can be reworded as guide RNA) recognizes and bind to the target DNA sequence, and Cas12a cleaves the target DNA bound to the complex. In this cleavage, when a reporter molecule defined by a fluorescent material and a quencher that are linked with a single-stranded DNA has been added to the reaction system in advance, Cas12a cleaves the single-stranded DNA acting as the linkage, and the fluorescent substance emits fluorescence, enabling detection of the target DNA (NPL 1). Incidentally, CRISPR is the acronym for clustered regularly interspaced short palindromic repeats.

### CITATION LIST

### Non Patent Literature

NPL 1 Janice S. Chen, et al., Science, 2018, Vol. 360, Issue 6387, pp. 436-439

The present inventors found an issue in NPL 1. The issue is that when the complex of Cas12a and guide RNA is stored in an aqueous liquid, the enzyme activity tends to decrease in a short period.

### SUMMARY OF INVENTION

Accordingly, the present invention provides a reagent that can maintain the enzyme activity of the complex of Cas protein and guide RNA in aqueous liquid.

The reagent according to the present invention includes complex particles, each including a particle and a complex bound to the particle, and an aqueous liquid in which the complex particles are dispersed. The complex includes a Cas protein and a guide RNA bound to the Cas protein.

The present invention also provides a method for detecting a target nucleic acid. The method includes: mixing a reagent with a sample containing a target nucleic acid, the reagent containing complex particles in which a complex including a Cas protein and a guide RNA bound to the Cas protein is bound to particles, and a reporter molecule containing a nucleic acid having a specific nucleotide sequence; allowing the complex to bind to the target nucleic acid, thereby cleaving the reporter molecule containing the nucleic acid having a specific nucleotide sequence; and detecting a change of luminescence emitted when the nucleic acid having a specific nucleotide sequence is cleaved.

Further features of the present invention will become apparent from the following description of exemplary embodiments with reference to the attached drawings.

### BRIEF DESCRIPTION OF DRAWINGS

FIG. 1 is a plot suggesting that particles with Cas12a have enzyme activity.

FIG. 2 is a plot comparing the activities of Cas12a-direct-binding particles and Cas12a-antibody particles.

FIG. 3 is a plot comparing the activities of Cas12a-antibody particles and Cas12a-nickel particles.

FIG. 4 is a plot comparing the activities of Cas12a-antibody particles and Cas12a-commercially available His tag antibody particles.

FIG. 5 is a plot comparing the activities of Cas12a-antibody particles and a Cas12a-crRNA aqueous solution.

FIG. 6 is a plot showing differences in enzyme activity among Cas12a-antibody particle samples with varying proportions of Cas12a in preparation.

FIG. 7 is a plot showing differences in enzyme activity among Cas12a-antibody particle samples with varying proportions of anti-His tag antibody in preparation.

FIG. 8 is a plot comparing the activities between Cas12a-antibody particles and Cas12a-antibody particles (with crRNA added afterward).

FIG. 9 is a plot showing changes in the activities of Cas12a-antibody particles and a Cas12a-crRNA aqueous solution.

FIG. 10 is a plot showing the effect of blocking in Cas12a-antibody particle preparation.

FIG. 11A is a plot showing the effect of sonication of Cas12a-antibody particles on enzyme activity.

FIG. 11B is a plot showing the effect of sonication of a Cas12a-crRNA aqueous solution on enzyme activity.

FIG. 12A is a plot showing the enzyme activities of Cas12a-antibody particle samples prepared without blocking agents.

FIG. 12B is a plot showing the enzyme activities of Cas12a-antibody particle samples prepared with a blocking agent.

FIG. 12C is a plot showing the enzyme activities of Cas12a-antibody particle samples prepared with another blocking agent.

FIG. 12D is a plot showing the enzyme activities of Cas12a-antibody particle samples prepared with another blocking agent.

FIG. 13A is a micrograph of Cas12a-antibody particles prepared without blocking agents, used for examining particle aggregation.

FIG. 13B is a micrograph of Cas12a-antibody particles prepared with a blocking agent, used for examining particle aggregation.

FIG. 13C is a micrograph of Cas12a-antibody particles prepared with another blocking agent, used for particle aggregation.

FIG. 13D is a micrograph of Cas12a-antibody particles prepared with another blocking agent, used for particle aggregation.

FIG. 14 is a plot showing the enzyme activities of Cas12a-antibody particle samples using different types of core particles.

### DESCRIPTION OF EMBODIMENTS

The present invention will be further described in specific embodiments. The invention is not, however, limited to the following embodiments.

In the following description, the term “binding” is used to describe binding modes not only of chemical bonds, such as covalent bonds and coordination bonds, but also other binding modes, including affinity interactions and surface adsorption and their combinations.

Reagent for Detecting Target Nucleic Acid

The reagent in the implementation of the present invention contains complex particles, each including a particle and a complex bound to the particle, and an aqueous liquid in which the complex particles are dispersed. The complex includes a Cas protein and a guide RNA bound to the Cas protein.

In the reagent disclosed herein, the complex including a Cas protein and a guide RNA bound to the Cas protein is bound to particles. This can reduce the likelihood that the hydration of Cas protein weakens intramolecular hydrogen bonds and van der Waals force. The reason is that the particles, which are close to the complex, hinder water molecules from accessing the Cas protein. In some embodiments of the reagent, a plurality of molecules of the complex are bound to the particles. In this instance, the concentration of the complex increases locally, further hindering water molecules from accessing the Cas protein. Accordingly, the decrease in enzyme activity of the complex (Cas protein) is reduced. Consequently, the reagent disclosed herein can be used for a long period to cleave a specific nucleotide sequence.

In general, when the protein concentration of a reagent is high, the enzyme activity is unlikely to decrease even in an aqueous liquid, but the reagent needs diluting before use. In contrast, in the reagent disclosed herein, the enzyme activity is unlikely to decrease even though the concentration of the complex of the Cas protein and the guide RNA is low. Accordingly, the reagent need not be diluted and is easy to use.

In an embodiment, the reagent may be used to detect a target nucleic acid. The reagent in such an embodiment further contains a reporter molecule containing a nucleic acid having a specific nucleotide sequence. When the complex binds to the target nucleic acid, the complex cleaves the nucleic acid having a specific nucleotide sequence of the reporter molecule to change the luminescence from the reporter molecule.

The term target nucleic acid refers to one that conjugates with the guide RNA of the complex.

When the guide RNA binds to a target nucleic acid, the complex cleaves the nucleic acid having a specific nucleotide sequence, exhibiting enzyme activity. When the reagent for detecting a target nucleic acid according to an embodiment is used, the guide RNA cleaves the nucleic acid of the reporter molecule, thereby changing the luminescence. Thus, the target nucleic acid is detected by such a luminescence change. The nucleic acid having a specific nucleotide sequence may be a single-stranded DNA or the like. The reporter molecule may be one producing fluorescence whose intensity after the complex cleaves the nucleic acid having a specific nucleotide sequence is higher than before the cleavage.

The reagent disclosed herein reduces the decrease in the enzyme activity of the complex (Cas protein) in aqueous liquids. Accordingly, when the reagent is used to detect a target nucleic acid, the sensitivity to the target nucleic acid is less likely to decrease, even in aqueous liquids.

In an embodiment, the reagent contains less than 1 mg of the complex per 1 mL of the reagent. Even in the reagent with such a low complex concentration, the enzyme activity is less likely to decrease.

Particle-Complex Binding at N-Terminus

In an embodiment, the particles and the complex may be bound by an amide linkage. This amide linkage is formed by the C═O derived from the carboxy group of each particle and an N—H bond derived from the amino group of the protein. That is, the particle and the complex form a structure with an amide linkage.

In some embodiments, the particles are bound to the complex at the amino terminus of the Cas protein of the complex. This is because the structure in which the particles are bound to the N-terminus of the Cas protein exhibits a smaller decrease in enzyme activity than other structures in which the particles are bound to an amino group other than the N-terminus of the Cas protein (for example, to the amino group of lysine), as demonstrated in an Example described later.

Particle-Complex Binding

In an embodiment, complex particles may be formed by binding the complex to the particles by amide linkages. This structure can be established by providing each particle with a carboxy group at the surface and forming an amide linkage between the carboxy group and an amino group of the Cas protein. In some embodiments, the amino group is the amino terminus (N terminus) of the Cas protein.

Cas Protein

In some embodiments, the Cas protein (CRISPR-associated proteins) may be Cas12 or Cas13, more specifically, Cas12a or Cas13a. The Cas protein used in the implementation of the present disclosure has an activity to cleave nucleic acid.

In some embodiments, Cas12 may be at least one selected from the group consisting of LbCas12a, AsCas12a, FnCas12a, and AaCas12b.

In some embodiments, Cas13 may be at least one selected from the group consisting of LwaCas13a, LbaCas13a, LbuCas13a, BzoCas13b, PinCas13b, PbuCas13b, AspCas13b, PsmCas13b, RanCas13b, PauCas13b, PsaCas13b, PinCas13b, CcaCas13b, PguCas13b, PspCas13b, PigCas13b, and Pin3Cas13b.

Guide RNA

In the implementation of the present disclosure, the guide RNA forms a complex with the Cas protein, as described above. Also, the guide RNA binds to (conjugates with) a target nucleic acid, allowing the complex to cleave the nucleic acid having a specific nucleotide sequence. The guide RNA is designed to bind to a target nucleic acid that is an indicator for diagnosing disease states. In other words, the guide RNA has a complementarity with a target nucleic acid sequence sufficient to hybridize with the specific nucleotide sequence of the target nucleic acid and induce the sequence-specific binding of the complex including the Cas protein and the guide RNA. The guide RNA may contain any polynucleotide sequence provided that it satisfies the above requirement. The guide RNA may also be referred to as crRNA.

Target Nucleic Acid

In some embodiments, the target nucleic acid may be DNA (deoxyribonucleic acid), RNA (ribonucleic acid), or the like. Such a target nucleic acid can be applied to diagnose disease states and diatheses. Disease states include cancer, autoimmune diseases, and infectious diseases. Pathogens of infectious diseases include, for example, DNA viruses and RNA viruses, but any target nucleic acid may be selected without limiting to DNA or RNA.

Reporter Molecule

The reporter molecule used in the implementation of the present disclosure may be a molecule from which the luminescence changes when the nucleic acid of the reporter protein is cleaved by the trans-cleavage reaction of the complex (Cas protein) and is otherwise not limited. The change of luminescence may be a change in intensity or wavelength of the luminescence. The reporter molecule may be one producing fluorescence whose intensity after the complex cleaves the nucleic acid having a specific nucleotide sequence is higher than before the cleavage.

For example, the reporter molecule may have a structure in which a fluorescent material producing fluorescence and a quencher material reducing the intensity of the fluorescence from the fluorescent material are bound via the above-described nucleic acid having a specific nucleotide sequence. The nucleic acid having a specific nucleotide sequence may be a single-stranded DNA, a double-stranded DNA, RNA, or the like. Any nucleotide sequence may be used as the specific nucleotide sequence of the nucleic acid, provided that it can be cleaved by the trans-cleavage reaction of the complex (Cas protein). In some embodiments, the nucleic acid having a specific nucleotide sequence includes 5 to 30 bases, for example, 10 or more bases.

In an embodiment using Cas12, the reporter molecule may have a structure in which a fluorescent material and a quencher are linked with a single-stranded DNA. Examples of such a molecule include the reporter molecule (hereinafter referred to as the IDT reporter) included in DNaseAlert Substrate Nuclease Detection System kit (produced by IDT, 11-02-01-04), a molecule in which a fluorescent material Cy5® and a quencher BHQ-3® are linked with DNA, a molecule in which a fluorescent material Alexa Fluor® 647 and a quencher Iowa Black® RQ-Sp are linked with DNA, a molecule in which a fluorescent material Alexa Fluor® 647 and a quencher BHQ-2® are linked with DNA, and a molecule in which a fluorescent material 6-FAM® and a quencher Iowa Black® FQ are liked by DNA. In an embodiment using Cas13, the reporter molecule may have a structure in which a fluorescent material and a quencher are linked with RNA.

The number of bases of the single-stranded DNA linking the fluorescent material and the quencher is not limited but may be 5 or more, depending on the selected fluorescent material and quencher. Reporter molecules other than those in a combination of a fluorescent material and a quencher may be used, including molecules in which a fluorescent material and biotin are kinked by a single-stranded nucleic acid. When the Cas protein cleaves the single-stranded nucleic acid linking a fluorescent material and biotin, biotin is released. Therefore, streptavidin solid-phased on paper or any other medium may not trap the reporter molecule. In such a case, a lateral flow technique capable of detecting the difference in mobility of fluorescent materials may be used.

Reaction Buffer

The reaction buffer used in the preparation of the reagent disclosed herein may be a Tris or HEPES buffer that has been reported about Cas12a and Cas13a enzyme reactions. Examples of such a buffer include NEBuffer™ 2.1 (10 mM Tris-HCl, 50 mM NaCl, 10 mM MgCl₂, 100 μg/mL BSA, pH 7.9), a binding buffer (20 mM Tris-HCl (pH 7.6), 100 mM KCl, 5 mM MgCl₂, 1 mM DTT, 5% glycerol, 50 μg/mL heparin), and FZ buffer (20 mM HEPES, 60 mM NaCl, 6 mM MgCl₂, pH 6.8).

The reaction temperature is set at about 37° C. in some embodiments, but other temperatures are acceptable. For example, EnGen™ Lba Cas12a (Cpf1) produced by NEW ENGLAND BioLabs is known to be active over a wider range, 16° C. to 48° C., than Asp Cas12a (Cpf1).

Linker (Linkage Between Cas Protein and Particles)

The linkage between the Cas protein and the particles may be referred to as the linker.

In an embodiment, each particle and the complex may be bound via one or more linkers.

The linker may include a peptide formed by consecutive 6 to 11 histidine residues and, in some embodiments, may include a peptide formed by consecutive 6 histidine residues (hereinafter also referred to as His tag). In this instance, the linker may include an antibody (anti-His tag antibody) that is bound to this peptide (His tag) by an antigen-antibody reaction. In such a structure, the linker may have a structure in which the C═O derived from the carboxy group of each particle and the N—H derived from an amino group of the antibody may form an amide linkage. This linker also includes a structure in which the antibody is bound to the His tag and a structure in which the His tag is bound to the Cas protein.

A metal complex that binds to this peptide (His tag) may be used as a linker, for example, a complex of nitrilotriacetic acid or iminodiacetic acid and a divalent nickel ion.

Examples of the binding position between the Cas protein and the linker on the protein side include the c-amino group of a lysine residue of the Cas protein, the a-amino group of the Cas protein N-terminus, or tag peptide sequences or tag proteins artificially inserted into the N terminus of the Cas protein. Desirably, the Cas protein and the linker are bound at a position not inhibiting the activity of the Cas Protein. For example, a position away from the carboxy terminus (C terminus) of the Cas protein is desirable because Cas proteins are known to have their active site on the C terminus side. For example, the N terminus is particularly desirable because it is away from the C terminus and into which various peptides or tag proteins can be inserted. Tag peptides include His tag, HA tag, and DDDDK tag (FLAG®).

The tag protein may be HaloTag®.

The binding position between the particles and the linker on the particle side varies depending on the type of linker. In an embodiment, when the linker is bound to the c-amino group of a lysine residue of the Cas protein or the a-amino group of the Cas protein N terminus, the particles can be bound to the linker at the carboxy or aldehyde group or the like of the particle. When an amino group of the Cas protein and the carboxy group of the particle form an amide linkage, for example, a condensation reaction of N-hydroxysuccinimide (NHS)/water-soluble carbodiimide (WSC) may be used. In another embodiment, when a His tag artificially inserted into the N terminus of the Cas protein is bound to the particles, an anti-His tag antibody may be used as a linker. In this instance, for example, the carboxy group of the particles is used as the linker on the particle side and at which the anti-His tag antibody is bound to the particle surfaces by a condensation reaction of an amino group of the anti-His tag antibody and NHS/WSC. Thus, the Cas protein and the particles can be bound to each other by an antigen-antibody reaction using the His tag at the N terminus of the Cas protein and the anti-His tag antibody. As an alternative to the use of anti-His tag antibody, the Cas protein may be immobilized on the particles by binding a metal chelating ligand, such as iminodiacetic acid (IDA) or nitrilotriacetic acid (NTA), to the particles as the linker and forming a coordination bond with a metal ion, such as nickel ion or cobalt ion.

Other possible linkers include linkages of a tag peptide sequence or tag protein and its affinity moiety, complexes of avidin and biotin, and polyethylene glycols with various functional groups at the terminals. The Cas protein and the particles may be bound by physical adsorption of the Cas protein to the particle surfaces.

Particles

The particles are not limited, provided that the complex can bind to the particles. In some embodiments, the particles each have the carboxy group, which is likely to bind to the complex or the linker. The primary shape of the particles may be perfectly spherical and can be in rods or sheets. Also, the particles may be in primary particles or secondary particles that are aggregates of the primary particles. The materials of the particles include polymer resin (styrene resin, acrylic resin, etc.), agarose-carrier resin, other resins, silica, metals, and latex. Examples of such particles include Magnosphere™ MS300, Magnosphere™ MS160, and PureProteome™ Nickel Magnetic Beads. In some embodiments, the particles contain paramagnetic, ferromagnetic, or supermagentic material, for example, iron, nickel, or magnetite. Other materials may be used. In an embodiment, magnetic particles may be used. Magnetic particles enable the position of the complex to be controlled by applying a magnetic field to the particles.

In some embodiments, the particles have diameters of 10 nm or more, for example, 1 μm to 10 μm. Specific examples of the particle size measurement method include observation under an optical or electron microscope, laser diffraction, dynamic light scattering, and centrifugal sedimentation, but other methods may also be used.

Blocking Agent

In an embodiment, the reagent may contain a blocking agent. When the particles and the Cas protein are bound, the blocking agent can fill the linker-bound portion of the particles to which the Cas protein is not bound. For example, when an amino group of the Cas protein or anti-His tag antibody is condensed with the carboxy group of the particles using NHS/WSC, the portion to which the Cas protein is not bound after the reaction can be reacted with ethanol amine or amino group-containing polyethylene glycol (PEG).

Method for Detecting Target Nucleic Acid

The method disclosed herein for detecting a target nucleic acid includes the following steps:
(1) mixing a reagent with a sample containing a target nucleic acid, the reagent containing complex particles in which a complex including a Cas protein and a guide RNA bound to the Cas protein is bound to particles, and a reporter molecule containing a nucleic acid having a specific nucleotide sequence;
(2) allowing the complex to bind to the target nucleic acid, thereby cleaving the nucleic acid having a specific nucleotide sequence; and
(3) detecting a change of luminescence emitted when the nucleic acid having a specific nucleotide sequence is cleaved.

The terms used in the target nucleic acid detection method disclosed herein are the same as in the above-described embodiments, and thus descriptions thereof are omitted.

The complex particles mentioned in step (1) may be formed by binding the Cas protein and the guide RNA to form the complex and then binding the resulting complex to particles. Alternatively, the complex particles may be formed by binding the Cas protein to particles and then binding guide RNA to that Cas protein.

### EXAMPLES

The present invention will be further described in detail with reference to Examples but is not limited to the following examples.

Preparation of Reagent

Before describing the Examples of the present invention, the materials used in the Examples and the method for evaluating test reagents will be described.

Adjustment of Cas12a Concentration

Using nuclease-free water (produced by NEB, B1500L, hereinafter referred to as water), 100 μM EnGen™ LbaCas12a (Cpf1) (produced by NEW ENGLAND BioLabs (NEB), M0653T) was diluted to adjust the concentration.

Adjustment of crRNA Concentration

For adjusting the crRNA concentration, 100 μM crRNA (LbaCas12a-crRNA1) (customized product, produced by SIGMA) for EnGen™ LbaCas12a (Cpf1) (produced by NEB, M0653T) was diluted with water. The sequence is as follows:

(Sequence Number 1)
uaauuucuacuaaguguagaugucuggccuuaauccaugcc

Preparation of DNA

Target DNA 113 bp was amplified by polymerase chain reaction (PCR) using a deoxyribonucleic acid (DNA) aqueous solution for quantitative analysis (1 ng/μL, 600 bp) (produced by FUJIFILM Wako Pure Chemical Corporation, 630-31991) as a template. After purification, the concentration was measured with a Qubit fluorometer. The sequence of the primer is as follows:

(Sequence Number 2)
Forward:
CTCACGCCTTATGACTG
(Sequence Number 3)
Reverse:
TAGCTATGAGGCATGGAT

The DNA 113 bp whose concentration had been measured was diluted with water to prepare 4 nM stock. The 4 nM stock was further diluted with water to prepare DNA solutions with varying concentrations.

The sequence of DNA 113 bp is as follows: The underlined portion indicates the target sequence for Cas12a.

(Sequence Number 4)
ctcacgccttatgactgcccttatgtcaccgcttatgtctcccgatatca
cacccgttatctcagccctaatctctgcggtttagtctggccttaatcca
tgcctcatagcta

Preparation of 12 μM Reporter Molecule

The reporter molecule (hereinafter referred to as the IDT reporter) included in DNaseAlert Substrate Nuclease Detection System kit (produced by IDT, 11-02-01-04) was used. The reporter molecule in 12 vials, each containing 50 pmol of the reporter molecule, was dissolved in 50 μL of 800 nM solution of HiLyte™ Fluor 488 (produced by AnaSpec, hereinafter referred to as HiLyte 488). HiLyte 488 was used as the standard fluorescent material in 96 wells.

Preparation of Reaction Buffer

NEBuffer™ 2.1 (10 mM Tris-HCl, 50 mM NaCl, 10 mM MgCl₂, 100 μg/mL BSA, pH 7.9) was used as the reaction buffer. More specifically, 10×NEBuffer™ 2.1, supplied with EnGen™ LbaCas12a (Cpf1) (produced by NEB, M0653T), was added in an amount of one-tenth of the reaction solution.

Experiments on Immobilization of Anti-His Tag Antibody to Magnetic Particles

Dispersion liquids of magnetic particles (Magnosphere™ MS300 and Magnosphere™ MS160) were each placed in microtubes, and the magnetic particles were settled down with a magnet. After removing the supernatant liquor, an MES buffer solution (100 mM, pH 5.4) was added into a pellet containing the magnetic particles to disperse the particles again and to which N-hydroxysulfosuccinimide (sulfo-NHS) and water-soluble carbodiimide (WSC) were added. After stirring at 25° C. for 1 hour, the magnetic particles were collected with a magnet. The collected magnetic particles were rinsed with an MES buffer solution and dispersed in the MES buffer solution and to which a desired amount of an anti-His tag antibody (Anti-His-tagmAb, produced by MBL Life Science) was added, followed by stirring at 25° C. for 2 hours. Then, only in the case of performing blocking operation, a largely excessive amount of PEG amine with a molecular weight of 5000 was added, followed by stirring at room temperature for 45 minutes. Irrespective of whether or not the blocking operation was performed, a largely excessive amount of ethanol amine was subsequently added to deactivate the active groups at the surfaces of the magnetic particles. The magnetic particles were collected with a magnet and rinsed with an MES buffer solution to yield antibody-immobilized beads. A storage buffer (10 mM HEPES-NaOH (pH 7.9), 50 mM KCl, 1 mM EDTA, 10% glycerol) was added to the antibody-immobilized beads to prepare an antibody particle liquid. Thus, samples of antibody particles were prepared and stored at 4° C. until being used.

Experiments on Reaction of Cas12a-crRNA with Antibody Particles

The diluted Cas12a and crRNA were mixed to a concentration ratio of 1:1.25, and the mixture was incubated at 37° C. for 30 minutes to produce a Cas12a-crRNA complex.

The antibody particle liquid (1 wt %) prepared above and a commercially available product of anti-His tag antibody particles (Anti-His-tag mAb-Magnetic Beads® (produced by MBL Life Science, 1 wt %) were each taken into 2 mL sample tubes (model number: 1-1600-04, manufactured by VIOLAMO). After stirring the particles with a Vortex mixer, the sample tubes were allowed to stand in a magnetic stand (Magical Trapper, model number: MGS-101, manufactured by TOYOBO) for 1 minute, followed by removing the liquid phase. PBS-T was added as a cleaning liquid for the particles. After stirring with a Vortex mixer, the liquid phase was removed. These operations were repeated twice. The particles were suspended in PBS-T, and the Cas12a-crRNA solution prepared above was added to desired concentrations. After stirring with a Vortex mixer, each mixture of the particles and the Cas12a-crRNA solution was subjected to a reaction in a shaker for 1 hour. After the reaction, the liquid phase was removed, and the resulting particles were rinsed with PBS-T. The particles were suspended in PBS-T and a storage buffer each to yield samples of Cas12a-antibody particles. After being stirred with a Vortex mixer, the samples were stored at 4° C. until being used.

Experiments on Reactions of Cas12a Alone with Antibody Particles and with crRNA

The antibody particle liquid (1 wt %) prepared above and a commercially available product of anti-His tag antibody particles (Anti-His-tag mAb-Magnetic Beads® (produced by MBL Life Science, 1 wt %) were each taken into 2 mL sample tubes (model number: 1-1600-04, manufactured by VIOLAMO). After stirring the particles with a Vortex mixer, the sample tubes were allowed to stand in a magnetic stand (Magical Trapper, model number: MGS-101, manufactured by TOYOBO) for 1 minute, followed by removing the liquid phase. PBS-T was added as a cleaning liquid for the particles. After stirring with a Vortex mixer, the liquid phase was removed. These operations were repeated twice. The particles were suspended in PBS-T, and Cas12a alone was added to desired concentrations. After stirring with a Vortex mixer, each mixture of the particles and Cas12a was subjected to a reaction in a shaker for 1 hour. After the reaction, the liquid phase was removed, and the resulting particles were rinsed with PBS-T. The particles were suspended in PBS-T and a storage buffer each to yield samples of Cas12a-antibody particles (without crRNA).

After removing the liquid phase, each sample of the Cas12a-antibody particles (without crRNA) was suspended in PBS-T. Then, crRNA (1.25 equivalent to Cas12a) was added, and the suspension was incubated at 37° C. for 30 minutes. After the reaction, the liquid phase was removed, and PBS-T was added. After stirring with a Vortex mixer, the liquid phase was removed. The resulting particles were suspended in PBS-T and a storage buffer each to yield samples of Cas12a-antibody particles (crRNA added afterward). After being stirred with a Vortex mixer, the samples were stored at 4° C. until being used.

Experiments on Reaction of Cas12a-crRNA with Nickel Particles

The diluted Cas12a and crRNA were mixed to a concentration ratio of 1:1.25, and the mixture was incubated at 37° C. for 30 minutes to produce a Cas12a-crRNA complex.

Commercially available nickel particles (PureProteome™ Nickel Magnetic Beads, produced by Merck, 3 wt %) were taken into 2 mL sample tubes (model number: 1-1600-04, manufactured by VIOLAMO). After stirring the particles with a Vortex mixer, the sample tubes were allowed to stand in a magnetic stand (Magical Trapper, model number: MGS-101, manufactured by TOYOBO), and the liquid phase was removed. PBS-T was added as a cleaning liquid for the particles. After stirring with a Vortex mixer, the liquid phase was removed. These operations were repeated twice. The particles were suspended in PBS-T, and the Cas12a-crRNA solution prepared above was added to desired concentrations. After stirring with a Vortex mixer, each mixture of the particles and the Cas12a-crRNA solution were subjected to a reaction in a shaker for 1 hour. After the reaction, the liquid phase was removed, and the resulting particles were rinsed with PBS-T. The particles were suspended in PBS-T and a storage buffer each to yield samples of Cas12a-nickel particles. After being stirred with a Vortex mixer, the samples were stored at 4° C. until being used.

Experiments on Immobilization of Cas12a-crRNA to Particles

The diluted Cas12a and crRNA were mixed to a concentration ratio of 1:1.25, and the mixture was incubated at 37° C. for 30 minutes to produce a Cas12a-crRNA complex. Dispersion liquids of magnetic particles (Magnosphere™ MS300 and Magnosphere™ MS160) were each placed in microtubes, and the magnetic particles were settled down with a magnet. After removing the supernatant liquor, an MES buffer solution (100 mM, pH 5.4) was added into a pellet containing the magnetic particles to disperse the particles again and to which N-hydroxysulfosuccinimide (sulfo-NHS) and water-soluble carbodiimide (WSC) were added. After stirring at 25° C. for 1 hour, the magnetic particles were collected with a magnet. The collected magnetic particles were rinsed with an MES buffer solution and dispersed in the MES buffer solution and to which a desired amount of the Cas12a-crRNA complex was added, followed by stirring at 25° C. for 2 hours. Then, only in the case of performing blocking operation, a largely excessive amount of PEG amine with a molecular weight of 5000 was added, followed by stirring at room temperature for 45 minutes. Irrespective of whether or not the blocking operation was performed, a largely excessive amount of ethanol amine was subsequently added to deactivate the active groups at the surfaces of the magnetic particles. The magnetic particles were collected with a magnet and rinsed with an MES buffer solution to yield Cas12a-immobilized particles. A storage buffer was added to the Cas12a-immobilized particles to yield a Cas12a-direct-binding particle liquid. Thus, samples of Cas12a-direct-binding particles were prepared and stored at 4° C. until being used.

Experiments on Immobilization of Cas12a Alone to Particles and Reaction with crRNA

Dispersion liquids of magnetic particles (Magnosphere™ MS300 and Magnosphere™ MS160) were each placed in microtubes, and the magnetic particles were settled down with a magnet. After removing the supernatant liquor, an MES buffer solution (100 mM, pH 5.4) was added into a pellet containing the magnetic particles to disperse the particles again and to which N-hydroxysulfosuccinimide (sulfo-NHS) and water-soluble carbodiimide (WSC) were added. After stirring at 25° C. for 1 hour, the magnetic particles were collected with a magnet. The collected magnetic particles were rinsed with an MES buffer solution and dispersed in the MES buffer solution and to which a desired amount of the Cas12a alone was added, followed by stirring at 25° C. for 2 hours. Then, only in the case of performing blocking operation, a largely excessive amount of PEG amine with a molecular weight of 5000 was added, followed by stirring at room temperature for 45 minutes. Irrespective of whether or not the blocking operation was performed, a largely excessive amount of ethanol amine was subsequently added to deactivate the active groups at the surfaces of the magnetic particles. The magnetic particles were collected with a magnet and rinsed with an MES buffer solution to yield Cas12a-immobilized beads. A storage buffer was added to the Cas12a-immobilized beads to prepare a Cas12a-direct-binding particle liquid (without crRANA). Thus, samples of Cas12a-direct-binding particles (without crRANA) were prepared and stored at 4° C. until being used.

After removing the liquid phase, Cas12a-direct-binding particles (without crRNA) were suspended in PBS-T. Then, crRNA (1.25 equivalent to Cas12a) was added, and the suspension was incubated at 37° C. for 30 minutes. After the reaction, the liquid phase was removed, and PBS-T was added. After stirring with a Vortex mixer, the liquid phase was removed.

The resulting particles were suspended in PBS-T and a storage buffer each to yield samples of Cas12a-direct-binding particles (crRNA added afterward), and the samples were stored at 4° C. until being used.

Measurement of Cas12a Enzyme Activity

Solutions of a complex were prepared by mixing 100 nM Cas12a solution and 100 nM crRNA solution in a volume ratio of 1:1.25 to desired Cas12a concentrations and incubating the mixture at 37° C. for 30 minutes. The solutions were used as Cas12a-crRNA aqueous solutions.

Different types of particles on which Cas12a is immobilized (Cas12a-antibody particles, Cas12a-directly binding particles, Cas12a-nickel beads, etc.) and the Cas12a-crRNA aqueous solutions were each placed to desired concentrations in a 96-well plate (manufactured by Thermo Fisher Scientific, 137101) and into which the target nucleic acid, NEBuffer, the reporter molecule, and water were added to a total volume of 80 μL. The fluorescent intensity of the resulting preparations was measured at 37° C. at intervals of 2 minutes for 1 to 2 hours with a fluorescent plate reader Synergy MX (manufactured by BioTek instruments). For the measurement wavelengths, HiLyte 488 was measured using an excitation wavelength of 485 nm±20 nm and a fluorescent wavelength of 528 nm±20 nm; and the IDT reporter was measured using an excitation wavelength of 535 nm±20 nm and a fluorescent wavelength of 595 nm±20 nm.

The following will describe each Example. Each reagent was prepared as described above.

### Example 1: Confirmation that Only Particles with Cas12a Exhibit Enzyme Activity

The trans-cleavage reaction of Cas12a-antibody particles (in the Figures, Beads with Cas12a binding via Ab) was evaluated from the changes with time in the fluorescence intensity of the IDT reporter relative to the fluorescence intensity of the internal standard dye HiLyte 488. The Cas12a-antibody particles were complex particles made up of a Cas12a-crRNA complex and particles, prepared by using the His tag introduced to the N terminus of LbaCas12a (cpf1, produced by NEB) and a linker, anti-His tag antibody (Anti-His-tag mAb, produce by MBL Life Science). For comparison, the changes in the fluorescence intensity of particles to which only the anti-His tag antibody was bound (Beads with Ab) and of only particles (MS300) are presented. Note that the particles of Cas12a-antibody particles are Magnosphere™ MS300 (produced by JSR) unless otherwise specified. The concentrations were 11.8 nM for Cas12a, 1 nM for the target DNA, 125 nM for the IDT reporter, and 8.3 nM for HiLyte 488. As the reaction buffer was used NEBuffer™ 2.1 (10 mM Tris-HCl, 50 mM NaCl, 10 mM MgCl₂, 100 μg/mL BSA, pH 7.9) (hereinafter referred to as the NEBuffer).

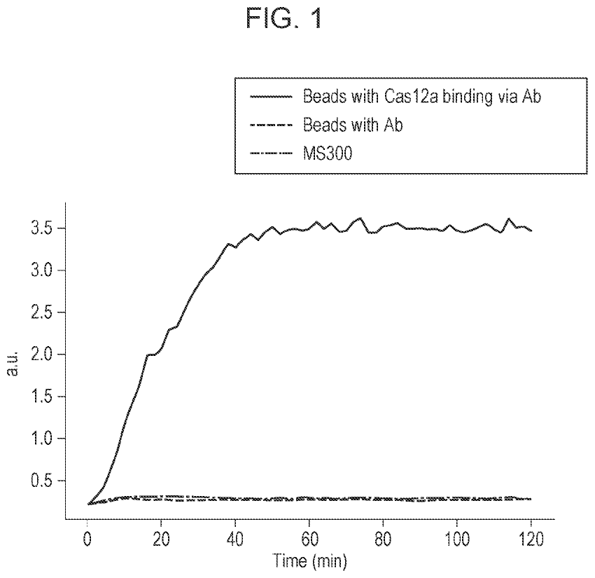

For the negative control (NC) against the Cas12a-antibody particles, the enzyme activities of antibody particles to which Cas12a was not bound and particles (Magnosphere™ MS300) to which neither Cas12a nor antibody was bound were compared. FIG. 1 shows the changes with time in the fluorescence intensity of the IDT reporter relative to the fluorescence intensity of the internal standard dye HiLyte 488 for each sample. The concentrations in the testing system were 11.8 nM for the Cas12a of the Cas12a-antibody particles, 1 nM for DNA 113 bp to be detected, 125 nM for the IDT reporter, and 8.3 nM for HiLyte 488. The amount of beads added to the testing system was 4 μL. The test results show that the NC samples, that is, antibody particles to which Cas12a was not bound and particles alone to which neither Cas12a nor antibody was bound, had no enzyme activity, and confirm that only Cas12a-antibody particles have an enzyme activity.

### Example 2: Comparison in Activity Between Cas12a-Direct-Binding Particles and Cas12a-Antibody Particles

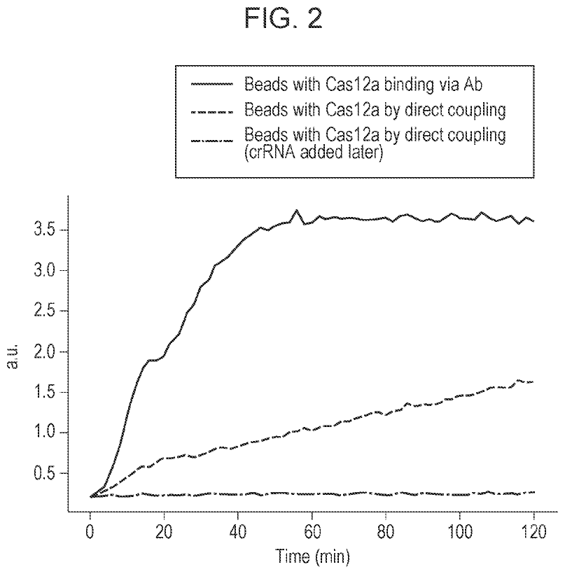

FIG. 2 shows a comparison in enzyme activity between different types of complex particles: Cas12a-direct-binding particles prepared by directly binding Cas12a to particles using the c-amino groups of a plurality of lysine residues at the surface of Cas12a and the carboxy groups at the surfaces of the particles; and Cas12a-antibody particles (denoted Beads with Cas12a binding via Ab in FIG. 2). The Cas12a-direct-binding particles presented in FIG. 2 are two types: one was produced by reacting a Cas12a-crRNA complex with particles (denoted as Beads with Cas12a by direct coupling in the Figure); and the other was produced by binding Cas12a alone to particles, followed by a reaction with crRNA (denoted as Beads with Cas12a by direct coupling (crRNA added later) in the Figure). For each of the samples, the changes with time in the fluorescence intensity of the IDT reporter relative to the fluorescence intensity of the internal standard dye HiLyte 488 are shown. As the particles of each sample were used Magnosphere™ MS300. The concentrations were 11.8 nM for Cas12a, 1 nM for the target DNA, 125 nM for the IDT reporter, and 8.3 nM for HiLyte 488. As the reaction buffer was used NEBuffer. The results are shown in FIG. 2.

The results suggest that Cas12-antibody particles (Case 1) have a high enzyme activity, whereas Cas12a-direct-binding particles (Case 2) have relatively low activity, and Cas12a-direct-binding particles (crRNA added afterward) (Case 3) hardly have enzyme activity.

The mechanism of Cas12a enzyme activity is known to be that insertion of crRNA and a target nucleic acid into Cas12a enables the Cas12a to exhibit the activity to cleave DNA and then randomly cleave the reporter molecule. Accordingly, the crRNA, the target nucleic acid, and the reporter molecule need to come close to the active site of Cas12a. Therefore, when the steric hindrance around the active site of Cas12a is large, the probability of those molecules coming close to the Cas12a decreases, and the Cas12a activity can probably decrease.

When Cas12a molecules are randomly bound to the particles without particular orientation, a certain percentage of the Cas12a molecules can be bound to the particles at the amino groups of the lysine residues near the active sites. Cas12a bound in such a manner has a large steric hindrance for the active site, and the enzyme activity of the Cas12a may have been lost. Accordingly, in binding modes having no orientation, a certain percentage of Cas12a molecules bound to the particles can probably be consistently deactivated.

In contrast, when Cas12a molecules are immobilized on the particles via the His tag at the N terminus of the Cas12a using anti-His tag antibody, nickel ions, or the like, the Cas12a molecules are bound to the particles with orientation. At this time, the distance between the N terminus and the active site of the Cas12a is large. This large distance reduces the steric hindrance around the active site of the Cas12a compared to the Cas12a-direct-binding particles. Consequently, the percentage of Cas12a molecules deactivated when immobilized on the particles is probably lower than that in the cases of direct binding.

These differences suggest that Cas12a-antibody particles and Cas12a-direct-binding particles differ in enzyme activity when their Cas12a concentrations are the same.

For the timing of forming the Cas12a-crRNA complex for Cas12a-direct-binding particles, the particles to which crRNA is added afterward exhibited relatively low enzyme activity. The reason for the low enzyme activity is probably that the structure of Cas12a changes when Cas12a is immobilized on the particle surfaces or that crRAN is not successfully introduced to the particle surfaces due to the steric hindrance or the like at the particle surfaces.

### Example 3: Comparison in Activity Between Cas12a-Antibody Particles and Cas12a-Nickel Particles

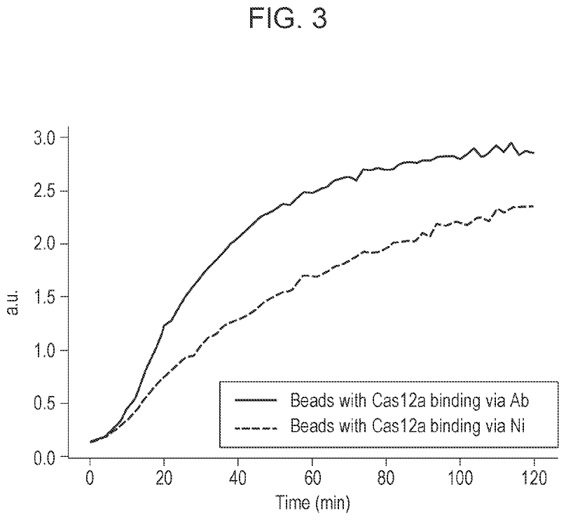

The His tag introduced to the N terminus of LbaCas12a (cpf1, produced by NEB) is known to form strong bonds with metal ions, such as nickel, cobalt, and copper ions. A Cas12a-nickel ion-particle complex (hereinafter referred to as Cas12a-nickel particles, denoted as Beads with Cas12a Binding via Ni in FIG. 3) was prepared using commercially available nickel ion coordination particles (PureProteome™ Nickel Magnetic Beads, produced by Merck, particle size: 10 μm). FIG. 3 shows the enzyme activity of this complex together with that of Cas12a-antibody particles (denoted as Beads with Cas12a Binding via Ab in FIG. 3, using Magnosphere™ MS300 (particle size: 3 μm) as the particles). For each of the samples, the changes with time in the fluorescence intensity of the IDT reporter relative to the fluorescence intensity of the internal standard dye HiLyte 488 are shown. The concentrations were 5.86 nM for Cas12a, 1 nM for the target DNA, 125 nM for the IDT reporter, and 8.3 nM for HiLyte 488. As the reaction buffer was used NEBuffer.

The results show that both the Cas12a-antibody particles and the Cas12a-nickel particles exhibited relatively high enzyme activity. Additionally, it is shown that the Cas12a immobilized on particles of 10 μm in diameter functions.

As with anti-His tag antibody, the nickel ion coordinates selectively with the His tag portion to form chelates. Accordingly, it is presumed that the binding of the enzyme to particles with orientation maintains high enzyme activity, like the experiments shown in FIG. 2.

### Example 4: Comparison in Enzyme Activity Between Cas12a-Antibody Particles and Cas12a-Commercially Available His Tag Antibody Particles

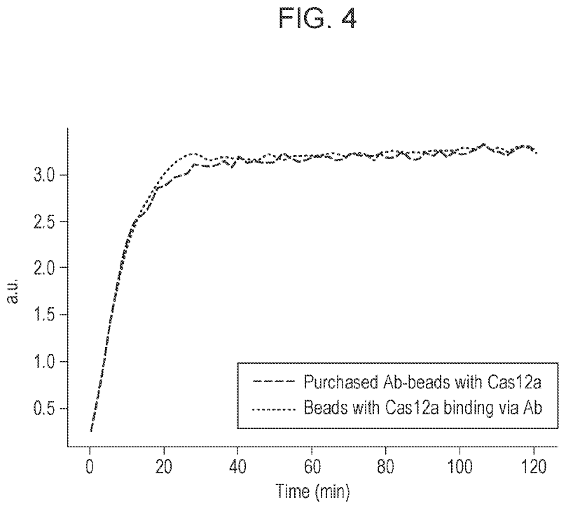

The enzyme activities of Cas12a-antibody particles (using Magnosphere™ MS160 (particle size: 1.5 μm), produced by JSR, as the particles, denoted as Beads with Cas12a binding via Ab in FIG. 4) and the complex particles (denoted as Purchased Ab-beads with Cas12a in FIG. 4) in which Cas12a-crRNA was bound to commercially available anti-His tag antibody particles (Anti-His-tag mAb-Magnetic Beads®, produced by MBL) were measured. The results are shown in FIG. 4. For each of the samples, the changes with time in the fluorescence intensity of the IDT reporter relative to the fluorescence intensity of the internal standard dye HiLyte 488 are shown. The concentrations were 50 nM for Cas12a, 1 nM for the target DNA, 125 nM for the IDT reporter, and 8.3 nM for HiLyte 488. As the reaction buffer was used NEBuffer.

The results show that Cas12a-antibody particles have the ability to bind the Cas12a-crRNA complex to the same degree as the Cas12a-commercially available His tag antibody particles and that Cas12a immobilized on particles of 1.5 μm in diameter functions.

### Example 5: Comparison in Enzyme Activity Between Cas12a-Antibody Particles and Cas12a-crRNA Aqueous Solution

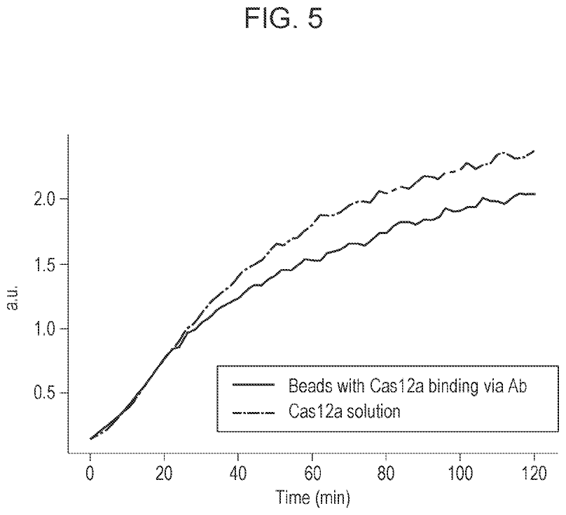

The enzyme activity of Cas12a-antibody particles (denoted as Beads with Cas12a binding via Ab in FIG. 5) and the enzyme activity of a Cas12a-crRNA complex in an aqueous solution (denoted as Cas12a solution in FIG. 5) were compared. For each of the samples, the changes with time in the fluorescence intensity of the IDT reporter relative to the fluorescence intensity of the internal standard dye HiLyte 488 are shown. The concentrations were 5 nM for Cas12a, 1 nM for the target DNA, 125 nM for the IDT reporter, and 8.3 nM for HiLyte 488. As the reaction buffer was used NEBuffer.

FIG. 5 is a comparison in enzyme activity between Cas12a-antibody particles and its positive control (PC), a Cas12a-crRNA aqueous solution, showing changes with time in the fluorescence intensity of the IDT reporter relative to the fluorescence intensity of the internal standard dye HiLyte 488 for each sample. In both the aqueous solution and the Cas-12a-antibody particles, the concentrations were 5 nM for Cas12a, 1 nM for DNA 113 bp to be detected, 125 nM for the IDT reporter, and 8.3 nM for HiLyte 488. The results show that the Cas12a-antibody particles had enzyme activity to the same degree as the Cas12a-crRNA aqueous solution (PC).

### Example 6: Differences in Enzyme Activity of Cas12a-Antibody Particles Among Preparations with Varying Cas12a Proportions

Four samples of Cas12a-antibody particles with different numbers of immobilized Cas12a molecules were prepared by varying the proportions of Cas12a to the antibody particles (Cas12a concentrations: 1.9E+6 molecules/particle (1.9×10⁶ molecules/particle), 9.0E+5 molecules/particle (9.0×10³ molecules/particle), 8.3E+6 molecules/particle (8.3×10⁶ molecules/particle), and 4.5E+6 molecules/particle (4.5×10⁶ molecules/particle)). These samples were subjected to an enzyme reaction, and their activities were measured. For each of the samples, the changes with time in the fluorescence intensity of the IDT reporter relative to the fluorescence intensity of the internal standard dye HiLyte 488 are shown. The concentrations in the testing systems (reaction systems) were 11.7 nM for Cas12a, 1 nM for the target DNA, 125 nM for the IDT reporter, and 8.3 nM for HiLyte 488. As the reaction buffer was used NEBuffer.

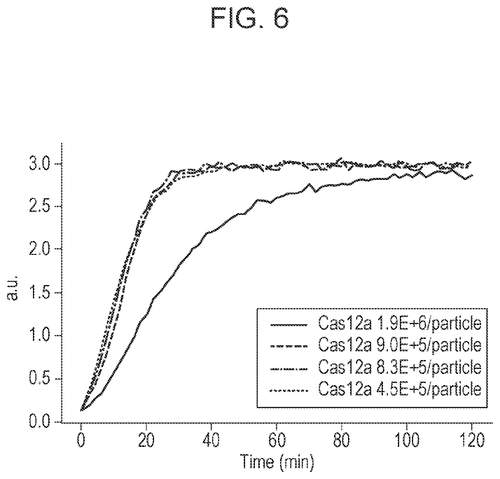

The enzyme activity was compared among the Cas12a-particle samples with different Cas12a-crRNA concentrations prepared by varying the concentration of Cas12a added for the reaction of Cas12a with antibody particles. FIG. 6 shows the changes with time in the fluorescence intensity of the IDT reporter relative to the fluorescence intensity of the internal standard dye HiLyte 488 for each sample.

The results demonstrate that the enzyme activity of Cas12a is not largely affected by the difference in the concentration of Cas12a in the preparation or the difference in the concentration of Cas12a on the particle surfaces.

### Example 7: Differences in Enzyme Activity of Cas12-Antibody Particles Among Preparations with Varying Anti-His Tag Proportions

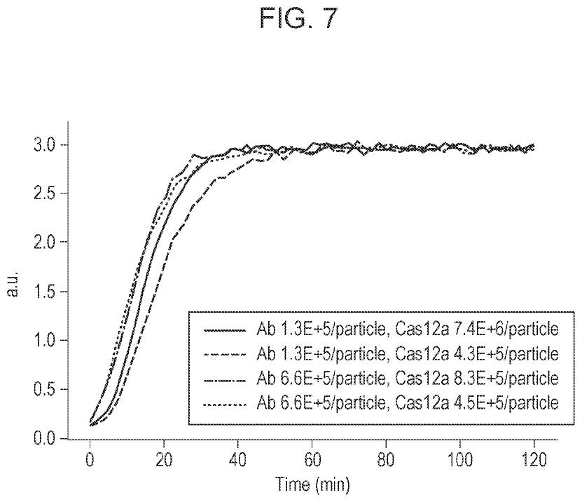

In preparation of Ca12a-antibody particle samples, the proportions of the anti-His tag antibody to the particles were varied. Also, the proportions of the Cas12a to the antibody particles were varied in the preparation of Cas12a-antibody particle samples (Cas12a concentrations of 4.3E+5/particle (4.3×10⁵/particle) and 7.4E+5/particle (7.4×10⁵/particle) to antibody concentration of 1.3E+5/particle (1.3×10⁵/particle); Cas12a concentrations of 4.5E+5/particle (4.5×10⁵/particle) and 8.3E+5/particle (8.3×10⁵/particle) to antibody concentration of 6.6E+5/particle (6.6×10⁵/particle)). These samples were subjected to an enzyme reaction, and their activities were measured. For each of the samples, the changes with time in the fluorescence intensity of the IDT reporter relative to the fluorescence intensity of the internal standard dye HiLyte 488 are shown. The concentrations were 11.7 nM for Cas12a, 1 nM for the target DNA, 125 nM for the IDT reporter, and 8.3 nM for HiLyte 488. As the reaction buffer was used NEBuffer. The enzyme activity was compared among the Cas12a-antibody particle samples prepared by varying the concentration of anti-His tag antibody in immobilizing anti-His tag antibody on particles. In each sample of Cas12a-antibody particles, the proportion of the Cas12a to the antibody particles was also varied. FIG. 7 shows the changes with time in the fluorescence intensity of the IDT reporter relative to the fluorescence intensity of the internal standard dye HiLyte 488 for each sample. The concentrations in each testing system (reaction system) were 11.7 nM for the Cas12a bound to particles, 1 nM for DNA 113 bp to be detected, 125 nM for the IDT reporter, and 8.3 nM for HiLyte 488.

The results demonstrate that the enzyme activity of Cas12a is not affected by the difference in the concentration of anti-His tag antibody in the preparation.

### Example 8: Comparison in Enzyme Activity Between Cas12a-Antibody Particles and Cas12a-Antibody Particles (crRNA Added Afterward)

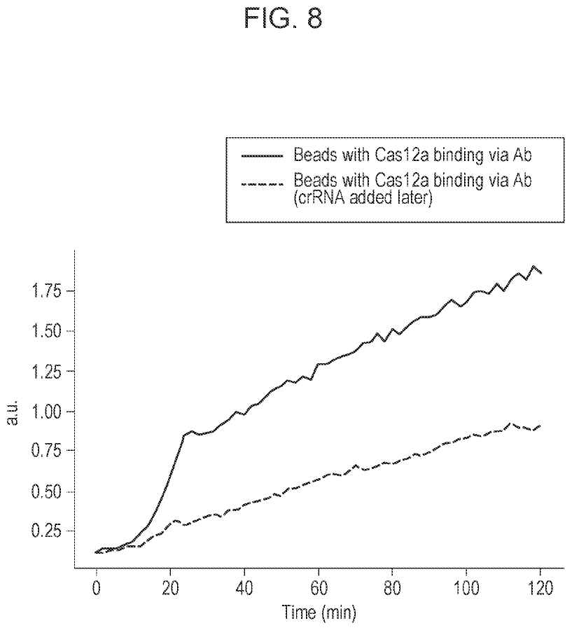

Two types of Cas12a-antibody particles were compared: one is Cas12a-antibody particles (denoted as Beads with Cas12a binding via Ab in FIG. 8) prepared by first forming a Cas12a-crRNA complex and then binding the complex to antibody particles; the other is Cas12a-antibody particles (crRNA added afterward) (denoted as Beads with Cas12a binding via Ab (crRNA added later) in FIG. 8) prepared by first binding Cas12a alone to antibody particles and then adding crRNA. For each of the samples, the changes with time in the fluorescence intensity of the IDT reporter relative to the fluorescence intensity of the internal standard dye HiLyte 488 are shown. The concentrations were 5.8 nM for Cas12a, 1 nM for the target DNA, 125 nM for the IDT reporter, and 8.3 nM for HiLyte 488. As the reaction buffer was used NEBuffer.

Two cases were compared: one is first forming the complex (binary complex) of Cas12a and crRNA, followed by a reaction with antibody particles (case 1); the other is reacting Cas12a with antibody particles without forming the complex in advance, followed by a reaction with crRNA (Case 2). FIG. 8 shows the changes with time in the fluorescence intensity of the IDT reporter relative to the fluorescence intensity of the internal standard dye HiLyte 488 for each sample. The concentrations in each testing system (reaction system) were 5.8 nM for the Cas12a bound to particles, 1 nM for DNA 113 bp to be detected, 125 nM for the IDT reporter, and 8.3 nM for HiLyte 488.

The results show that the enzyme activity of the Cas12a-antibody particles (crRNA added afterward) was lower than that of the Cas12a-antibody particles. The reason for the low enzyme activity of the Cas12a-antibody particles to which crRNA was added afterward is probably that the structure of Cas12a changes when the Cas12a is immobilized on the particle surfaces or that crRAN is not successfully introduced to the particle surfaces due to the steric hindrance or the like at the particle surfaces.

### Example 9: Changes in Enzyme Activity of Cas12a-Antibody Particles and a Cas12a-crRNA Aqueous Solution

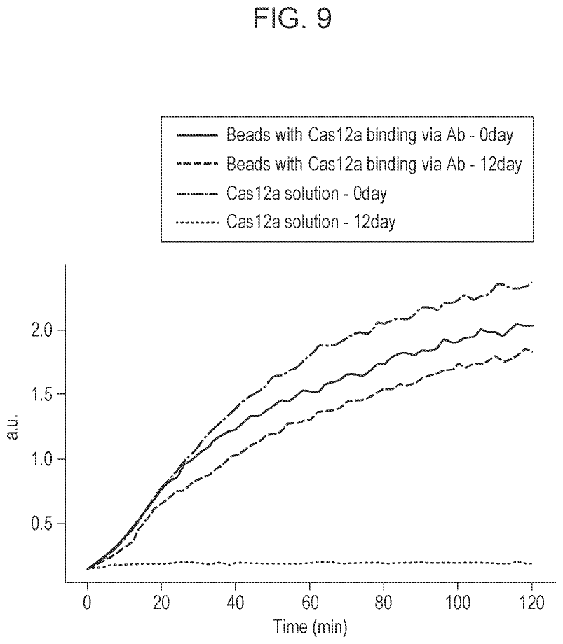

The stability of the enzyme activities of Cas12a-antibody particles (denoted as Beads with Cas12a binding via Ab in FIG. 9) and a Cas12a-crRNA aqueous solution (denoted as Cas12a solution in FIG. 9) was measured in their different states. The Cas12a-antibody particles and the Cas12a-crRNA aqueous solution were each adjusted to a Cas12a concentration of 100 nM and stored at 4° C. for 12 days. FIG. 9 shows the comparison in residual enzyme activity of the Cas12a in the samples 0 days and 12 days after the concentration was adjusted. For each of the samples, the changes with time in the fluorescence intensity of the IDT reporter relative to the fluorescence intensity of the internal standard dye HiLyte 488 are shown. The concentrations were 5 nM for final Cas12a, 1 nM for the target DNA, 125 nM for the IDT reporter, and 8.3 nM for HiLyte 488. As the reaction buffer was used NEBuffer.

The Cas12a concentration of the Cas12a-antibody particles and the Cas12a-crRNA aqueous solution was adjusted to 100 nM, and the samples were stored at 4° C. for 12 days, and each was compared to the enzyme activity on day 0. FIG. 9 shows the changes with time in the fluorescence intensity of the IDT reporter relative to the fluorescence intensity of the internal standard dye HiLyte 488 for each sample. The concentrations in each testing system (reaction system) were 5 nM for the Cas12a of both the Cas12a-crRNA aqueous solution and the Cas12a-antibody particles, 1 nM for DNA 113 bp to be detected, 125 nM for the IDT reporter, and 8.3 nM for HiLyte 488.

The results show that the enzyme activity of the Cas12a-crNMR aqueous solution stored for 12 days was deactivated, whereas the Cas12a-antibody particles substantially maintained the enzyme activity in 12-day storage. One of the reasons for this is probably that dense immobilization of Cas12a on particle surfaces maintained locally high Cas12a concentration.

### Example 10: Effect of Blocking in Preparation of Cas12a-Antibody Particles

The effect of a blocking agent on the immobilization of the anti-His tag antibody in the preparation of Cas12a-antibody particles was examined. FIG. 10 shows the changes with time in the fluorescence intensity of the IDT reporter relative to the fluorescence intensity of the internal standard dye HiLyte 488 for each sample. As the blocking agent was used amino group-containing PEG. The concentrations were 11.7 nM for the Cas12a bound to particles, 1 nM for DNA 113 bp to be detected, 125 nM for the IDT reporter, and 8.3 nM for HiLyte 488. The results suggest that the blocking agent used in the immobilization of the anti-His tag antibody does not affect the enzyme activity.

### Example 11: Effect of Sonication of Cas12a-Antibody Particles and Cas12a-crRNA Aqueous Solution on Enzyme Activity

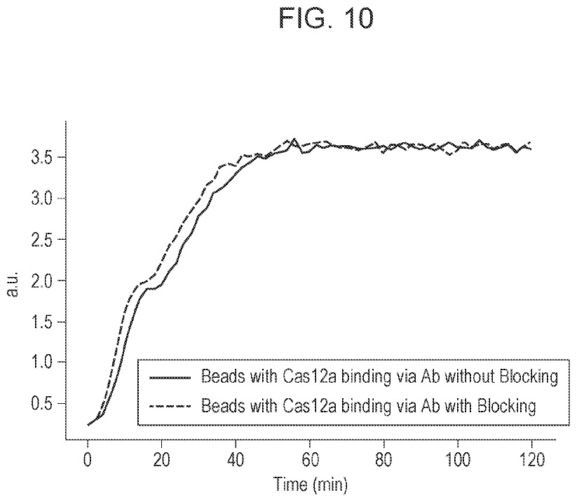

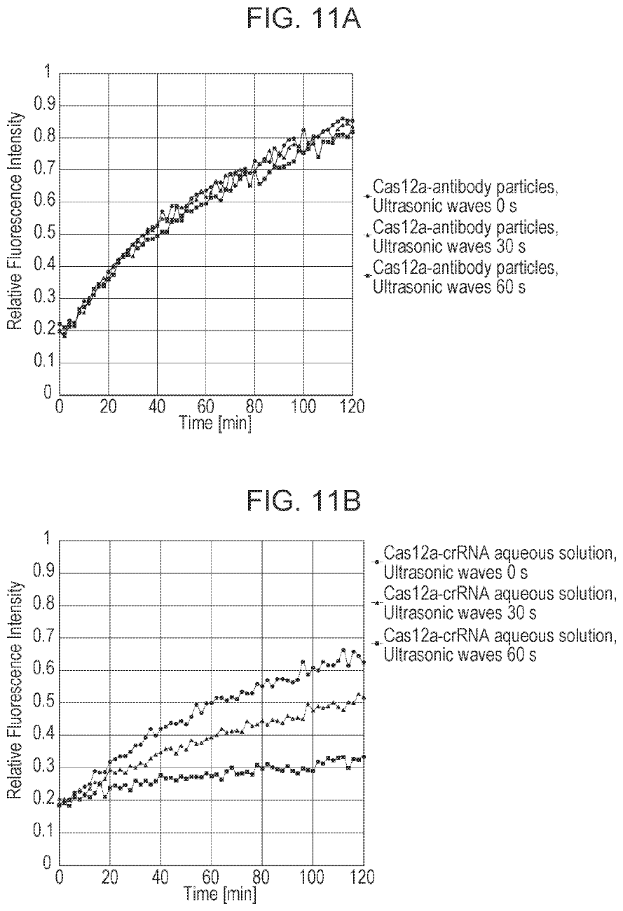

The effect of sonication of Cas12a-antibody particles and a Cas12a-crRNA aqueous solution on enzyme activity was examined. Cas12a-antibody particles and a Cas12a-crRNA aqueous solution were each added to a 1.5 mL tube (manufactured by Eppendorf) to a final Cas concentration of 10 nM, and with which DNA 113 bp to be detected was mixed to a final concentration of 2 nM, followed by incubation at 37° C. for 30 minutes. The resulting samples were subjected to sonication at 28 kHz in an ultrasonic cleaner (manufactured by AS ONE Corporation) filled with ice-cold water for 0 s, 30 s, and 60 s. Then, the enzyme activity was measured. FIG. 11A shows the results of Cas12a-antibody particles, and FIG. 11B shows the results of the Cas12a-crRNA aqueous solution.

FIGS. 11A and 11B each show the changes with time in the fluorescence intensity of the IDT reporter relative to the fluorescence intensity of the internal standard dye HiLyte 488. As the blocking agent was used amino group-containing PEG with a molecular weight of 5000. The concentrations were 5 nM for Cas12a bound to particles, 1 nM for DNA 113 bp to be detected, 125 nM for the IDT reporter, and 8.3 nM for HiLyte 488. The results demonstrate that the enzyme activity of the Cas12a-crNMR aqueous solution is deactivated by sonication, whereas the Cas12a-antibody particles maintain the enzyme activity.

### Example 12: Deference in Effect of Blocking Agents Used in Preparation of Cas12a-Antibody Particles

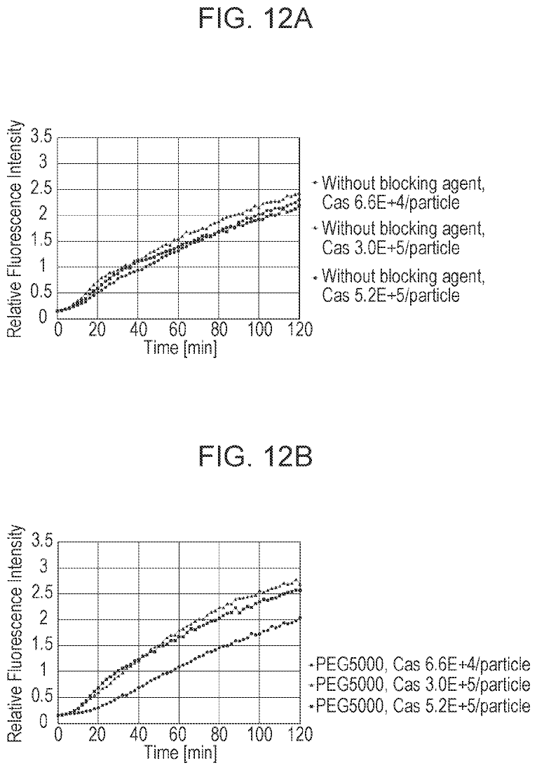

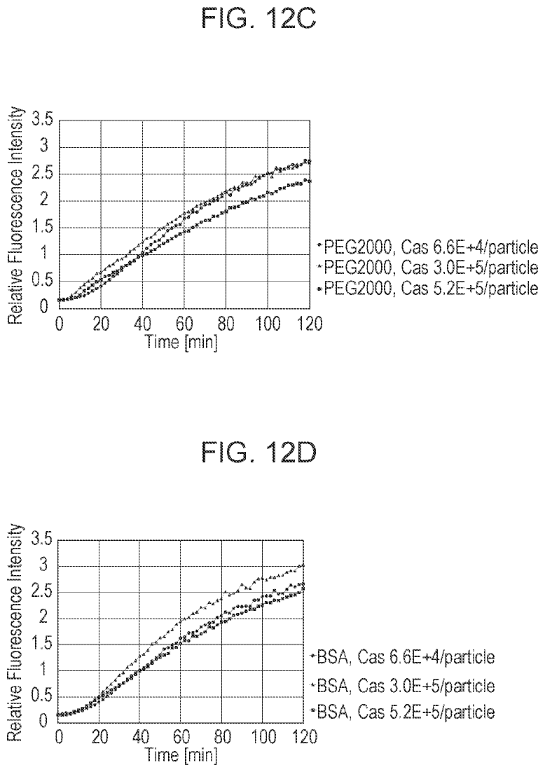

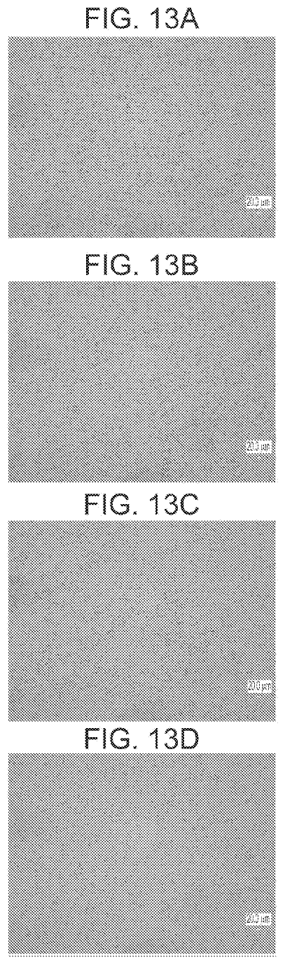

The difference in effect of blocking agents used in immobilization of the anti-His tag antibody in the preparation of Cas12a-antibody particles was examined. As the blocking agent, amino group-containing PEGs having molecular weights of 5000 and 2000, respectively, and bovine serum albumin (BSA) were used. For the samples using no blocking agent, the enzyme activity and particle aggregation were examined. The number of immobilized Cas molecules was varied between about 6E+4 molecules/particle (6×10⁴ molecules/particle) and about 8E+5 molecules/particle (8×10³ molecules/particle), and thus three types of samples were prepared for each blocking agent. FIGS. 12A to 12D show enzyme activities represented by the changes with time in the fluorescence intensity of the IDT reporter relative to the fluorescence intensity of standard dye HiLyte 488. FIG. 12A shows the results of samples using no blocking agent; FIG. 12B shows the results of samples using PEG having a molecular weight of 5000 as the blocking agent; FIG. 12C shows the results of samples using PEG having a molecular weight of 2000 as the blocking agent; and FIG. 12D shows the results of samples using BSA as the blocking agent. The concentrations were 4 nM for Cas12a bound to particles, 1 nM for DNA 113 bp to be detected, 125 nM for the IDT reporter, and 8.3 nM for HiLyte 488. The results suggest that the samples using any one of the blocking agents exhibit good enzyme activity. For particle aggregation, FIGS. 13A to 13D show optical micrographs of Cas12a-antibody particle samples on a slide glass. In each Cas12a-antibody particle sample, the number of immobilized Cas was about 3E+5/particle (3×10⁵/particle). FIG. 13A shows the micrograph of the sample using no blocking agent; FIG. 13B shows the micrograph of the samples using PEG having a molecular weight of 5000 as the blocking agent; FIG. 12C shows the micrograph of the sample using PEG having a molecular weight of 2000 as the blocking agent; and FIG. 12D shows the micrograph of the sample using BSA as the blocking agent. Only the sample using no blocking agent exhibited an aggregation of the particles, suggesting that blocking agents have the effect of reducing particle aggregation.

### Example 13: Difference in the Enzyme Activity of Cas12a-Antibody Particles Between the Types of Core Particles

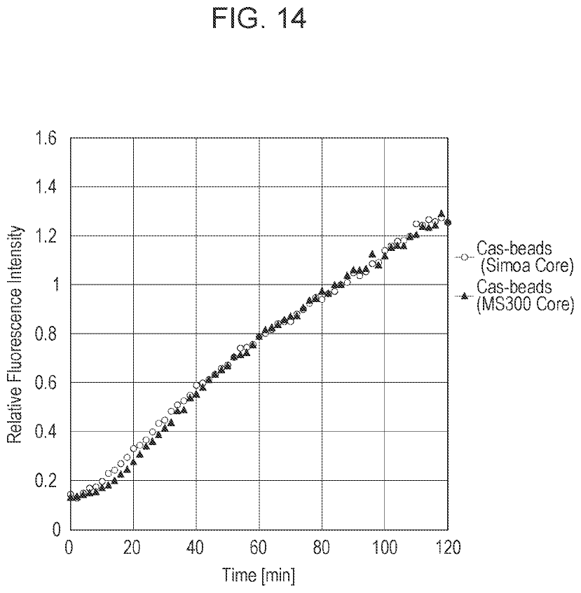

The core particles of the Cas12a-antibody particles were changed. As the core particles were used magnetic particles (produced by Quanterix Corp., 103611) included in Simoa® Homebrew Assay Starter Kit. EDC (1-ethyl-3-(3-dimethylaminopropyl)carbodiimide hydrochloride) (produced by Thermo Fisher Scientific) dissolved in Bead Conjugation Buffer (produced by Quanterix Corp.) was added to the magnetic particles subjected to buffer exchange to Bead Conjugation Buffer, followed by stirring at 4° C. for 30 minutes. Then, the magnetic particles were collected by a magnet. The collected magnetic particles were dispersed in Bead Conjugation Buffer and into which a desired amount of an anti-His tag antibody (Anti-His-tagmAb, produced by MBL Life Science) was added, followed by stirring at 4° C. for 2 hours. After rinsing the magnetic particles with Bead Wash Buffer (produced by Quanterix Corp.), Bead Blocking Buffer (produced by Quanterix Corp.) was added, followed by stirring at room temperature for 45 minutes. The magnetic particles were collected with a magnet and rinsed with Bead Wash Buffer. Then, Bead Diluent (produced by Quanterix Corp.) was added, followed by storage at 4° C. until being used. The resulting antibody particles were subjected to buffer exchange to PBS-T buffer, and a Cas12a-crRNA solution was added to a desired concentration. After stirring with a Vortex mixer, the particles and the Cas12a-crRNA solution were reacted in a shaker for 1 hour. After the reaction, the liquid phase was removed to yield Cas12a-antibody particles, followed by being suspended in pure water. The enzyme activity of the resulting Cas12a-antibody particles was measured. FIG. 14 shows the changes with time in the fluorescence intensity of the IDT reporter relative to the fluorescence intensity of the internal standard dye HiLyte 488. For comparison, the result of another type of Cas12a-antibody particles prepared using Magnosphere™ MS300 as the core particles and PEG having a molecular weight of 2000 as the blocking agent was also presented in FIG. 14.

The concentrations were 4 nM for Cas12a bound to particles, 1 nM for DNA 113 bp to be detected, 125 nM for the IDT reporter, and 8.3 nM for HiLyte 488. The results suggest that Cas12a-antibody particles having enzyme activity can be produced using different types of core particles.

The reagent according to the present invention can reduce the prompt decrease in the enzyme activity of Cas protein-guide RNA complexes in aqueous liquid.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. A reagent comprising:
complex particles, each including a particle and a complex bound to the particle; and an aqueous liquid in which the complex particles are dispersed,
wherein the complex includes a Cas protein and a guide RNA bound to the Cas protein.

2. The reagent according to claim 1, further comprising a reporter molecule containing a nucleic acid having a specific nucleotide sequence, the reporter molecule emits luminescence that changes when the complex to which a target nucleic acid is bound cleaves the nucleic acid having a specific nucleotide sequence, wherein the reagent acts as a reagent for detecting the target nucleic acid.

3. The reagent according to claim 2, wherein the change of luminescence is a change in intensity or wavelength of the luminescence.

4. The reagent according to claim 2, wherein the reporter molecule emits fluorescence whose intensity after the complex cleaves the nucleic acid having a specific nucleotide sequence is higher than before the cleavage.

5. The reagent according to claim 1, wherein the complex particles each contain a carboxy group.

6. The reagent according to claim 1, wherein the particles are bound to the complex at the amino terminus of the Cas protein.

7. The reagent according to claim 1, wherein each of the particles and the complex are bound by an amide linkage.

8. The reagent according to claim 1, wherein each of the particles and the complex are bound via a linker.

9. The reagent according to claim 8, wherein the linker includes a peptide formed by consecutive 6 to 11 histidine residues.

10. The reagent according to claim 9, wherein the linker includes an antibody that binds to the peptide by an antigen-antibody reaction.

11. The reagent according to claim 9, wherein the linker includes a metal complex that binds to the peptide.

12. The reagent according to claim 11, wherein the metal complex is a complex of nitrilotriacetic acid or iminodiacetic acid and a divalent nickel ion.

13. The reagent according to claim 8, wherein the linker includes polyethylene glycol.

14. The reagent according to claim 8, wherein the linker includes a complex of biotin and avidin.

15. The reagent according to claim 1, wherein the particles have a particle size of 1 μm to 10 μm.

16. The reagent according to claim 1, wherein the reagent contains less than 1 mg of the complex per 1 mL thereof.

17. The reagent according to claim 2, wherein the reporter molecule has a structure in which a fluorescent material emitting fluorescence and a quencher material reducing the intensity of the fluorescence from the fluorescent material are bound via the nucleic acid having a specific nucleotide sequence.

18. The reagent according to claim 1, further comprising a blocking agent.

19. The reagent according to claim 1, wherein the Cas protein is Cas12 or Cas13.

20. A method for detecting a target nucleic acid, the method comprising:
mixing a reagent with a sample containing a target nucleic acid, the reagent containing complex particles in which a complex including a Cas protein and a guide RNA bound to the Cas protein is bound to particles, and a reporter molecule containing a nucleic acid having a specific nucleotide sequence;
allowing the complex to bind to the target nucleic acid, thereby cleaving the reporter molecule containing the nucleic acid having a specific nucleotide sequence; and
detecting a change of luminescence emitted when the nucleic acid having a specific nucleotide sequence is cleaved.
